(19) United States
(12) Patent Application Publication
(10) Pub. No.: US 2003/0059485 A1
(43) Pub. Date: Mar. 27, 2003

(54) Treatment of allergic rhinitis

(76) Inventor: Ching-Hsiang Hsu, Taichung (TW)

Correspondence Address: FISH & RICHARDSON PC, 225 FRANKLIN ST, BOSTON, MA, 02110, US

(21) Appl. No.: 10/252,251

(22) Filed: Sep. 23, 2002

Related U.S. Application Data
(60) Provisional application No. 60/324,761, filed on Sep. 25, 2001.
(60) Provisional application No. 60/350,004, filed on Jan. 17, 2002.

(51) Int. Cl. A61K 35/78

(52) U.S. Cl. 424/725; 424/764; 424/769

(57) ABSTRACT

A nutraceutical composition containing extracts of at least three or four herbs, wherein the first herb, which is optional, is Hedysarum polybotrys Hand.-Mazz. or Astragalus membranceus Bge., the second herb is Atractylodes macrocephala kodiz, the third herb is Ledebouriella seseloides (hoffm.) Wolff, and the fourth herb is an anti-allergy herb. Also disclosed is a method of treating a disorder related to excessive secretion of histamine or interleukin-4 by using the extracts.

### RELATED APPLICATIONS

This application claims priority to U.S. Provisional Application Serial No. 60/324,761, filed Sep. 25, 2001 and U.S. Provisional Application Serial No. 60/350,004, filed Jan. 17, 2002, the contents of which are incorporated herein by reference.

### BACKGROUND OF THE INVENTION

Chinese herbs have long been used to treat different allergic and immunologic diseases. Yu Ping Feng San, a Chinese medicine, includes Astragalus membranceus Bge. (“Huang-Qi” in Chinese), Atractylodes macrocephala kodiz. (“Bai-Zhu” in Chinese), and Ledebouriella seseloides (hoffm.) Wolff (“Fang-Feng” in Chinese). In various formulas (i.e., different weight ratios of the three components), it has been used clinically to treat allergic rhinitis. However, their efficacy has not been entirely satisfactory.

### SUMMARY OF THE INVENTION

One aspect of this invention relates to a nutraceutical composition containing extracts of at least three or four herbs, wherein the first herb, which is optional, is Hedysarum polybotrys Hand.-Mazz. or Astragalus membranceus Bge. (e.g., its root), the second herb is the root of Atractylodes macrocephala kodiz (e.g., its root), the third herb is Ledebouriella seseloides (hoffm.) Wolff (e.g., its root), and the fourth herb is an anti-allergy herb. An anti-allergy herb is one that, when used alone or in combination with other herbs, can exhibit activity in treating allergy. The weight percentage of the herbal extracts in the nutraceutical composition ranges from 1% to 99%.

In one subset of the nutraceutical compositions, the fourth herb is Centipeda minimal (L.) A. Br. Et Aschers (e.g., its whole plant, “E-Bu-Shi-Cao” in Chinese). They include those wherein the weight ratio of the first, second, third, and fourth herbs is (0-1.5):(0.5-1.5):(0.5-1.5):(0.5-1.5). As indicated by this weight ratio, the first herb may not be included. In other words, the nutraceutical compositions may contain extracts of only three herbs.

The term “weight” refers to “dry weight,” i.e., the weight measured after the herb have been harvested and dried. Typically, the drying processes are specified by the regulations in herb-producing countries (e.g., P.R. China). Herbs so-obtained are suitable for transportation and long term storage.

In another subset of the nutraceutical compositions, the fourth herb is Magnolia lilifora Desr. (e.g., its flower; “Xin-Yi-Hua” in Chinese). They include those wherein the weight ratio of the first, second, third, and fourth herbs is (0-1.5):(0.5-1.5):(0.5-1.5):(0.5-1.5); and those containing extracts of four herbs.

In still another subset of the nutraceutical compositions, each composition contains extracts of exactly three or four herbs. The weight ratio of the first, second, third, and fourth herbs can be (0-1.5):(0.5-1.5):(0.5-1.5):(0.5-1.5).

Another aspect of this invention relates to a method of treating a disorder related to excessive secretion of histamine or interleukin-4. The method includes administering to a subject in need thereof a nutraceutical composition containing extracts of at least three or four herbs, wherein the first herb, which is optional, is Hedysarum polybotrys Hand.-Mazz. or Astragalus membranceus Bge. (e.g., the root), the second herb is Atractylodes macrocephala kodiz. (e.g., the root), the third herb is Ledebouriella seseloides (hoffm.) Wolff (e.g., the root), and the fourth herb is an anti-allergy herb. Examples of the disorder to be treated include, but are not limited to, allergic rhinitis, asthma, and eczyma.

The compositions used in the method of treating such a disorder include those wherein the fourth herb is Centipeda minimal (L.) A. Br. Et Aschers (whole plant) or Magnolia lilifora Desr (flower); those wherein the weight ratio of the first, second, third, and fourth herbs is (0-1.5):(0.5-1.5):(0.5-1.5):(0.5-1.5); and those containing extracts of three or four herbs.

The details of the invention are set forth in the accompanying description below. Other features, objects, and advantages of the invention will be apparent from the description and from the claims.

### DETAILED DESCRIPTION OF THE INVENTION

This invention is based on the discovery of an improved herbal medicine. The improved herbal medicine includes extracts of at least three or four herbs, wherein the first herb, which is optional, is the root of Hedysarum polybotrys Hand.-Mazz. or Astragalus membranceus Bge., the second herb is the root of Atractylodes macrocephala kodiz., the third herb is the root of Ledebouriella seseloides (hoffm.) Wolff, and the fourth herb is an anti-allergy herb.

Accordingly, a nutraceutical composition of this invention includes extracts of the root of Hedysarum polybotrys Hand.-Mazz. or Astragalus membranceus Bge., the root of Atractylodes macrocephala kodiz., the root of Ledebouriella seseloides (hoffm.) Wolff, and at least a fourth herb which is an anti-allergy herb. Examples of the fourth herb include, but are not limited to, the whole plant of Centipeda minimal (L.) A. Br. Et Aschers and the flower of Magnolia lilifora Desr. The weight ratio of the extracts of the first, second, third, and fourth herbs can be in the range of (0-1.5):(0.5-1.5):(0.5-1.5):(0.5-1.5). Preferred ratios can be determined by efficacy-evaluating methods described below or analogous methods. The nutraceutical composition is unexpectedly effective for the treatment of disorders related to excessive secretion of histamine or interleukin-4, such as allergic rhinitis, asthma, and eczyma.

The extracts of the herbs can be obtained by methods well known in the art. For instance, each herb is first soaked in water and then heated (e.g., at 100° C.), or first soaked in a mixture of water and an organic solvent (e.g., ethanol) and then heated (e.g., at 55° C.) for an extended period of time (e.g., 1-4 hours). The liquid phase, which contains active ingredients from the herbs, is then collected. The solvent (or the solvents) of the solution thus obtained, i.e., water (or a mixture of water and the organic solvent), is then removed under reduced pressure, yielding a residue (i.e., extracts of the herbs). The herbs can be handled together or individually to obtain their extracts.

The extracts thus obtained can be used to formulate a nutraceutical composition for treating (including preventing or ameliorating the symptoms of) a disorder related to excessive secretion of histamine or interleukin-4, such as allergic rhinitis, asthma, and eczyma. The nutraceutical composition can be a dietary supplement (e.g., a capsule or tablet, or placed in a mini-bag), a food product (e.g., a soft drink, milk, juice, or confectionary, or placed in a herbal tea-bag), or a botanical drug. The botanical drug can be in a form suitable for oral use, such as a tablet, a hard or soft capsule, an aqueous, or a syrup; or in a form suitable for parenteral use, such as an aqueous propylene glycol solution, or a buffered aqueous solution. The amounts of the active ingredients in the nutraceutical composition depend to a large extent on a subject's specific need. The amount will also vary, as recognized by those skilled in the art, depending on administration route, and possible co-usage of other agents useful for treating the above-mentioned disorders.

Herbal extracts thus obtained in an effective amount can also be formulated with a pharmaceutically acceptable carrier into a pharmaceutical composition for treating the above-mentioned disorders. “An effective amount” refers to the amount of the extracts which is required to confer therapeutic effect on the treated subject. Effective doses will vary, as recognized by those skilled in the art, depending on the route of administration, the excipient usage, and the optional co-usage with other therapeutic treatments. Examples of pharmaceutically acceptable carriers include colloidal silicon dioxide, magnesium stearate, cellulose, sodium lauryl sulfate, and D&C Yellow # 10.

The herbal extracts can be formulated into dosage forms for different administration routes utilizing conventional methods. For example, they can be formulated in a capsule, a gel seal, or a tablet for oral administration. Capsules may contain any standard pharmaceutically acceptable materials such as gelatin or cellulose. Tablets may be formulated in accordance with conventional procedures by compressing mixtures of the polysaccharide with a solid carrier and a lubricant. Examples of a suitable solid carrier include starch and sugar bentonite. The herbal extracts can also be administered in the form of a hard shell tablet or a capsule containing a binder, e.g., lactose or mannitol, a conventional filler, and a tableting agent. The pharmaceutical composition may be administered via a parenteral route, e.g., topically, intraperitoneally, and intravenously. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic compound.

Also within the scope of this invention is use of the herbal extracts described above for treating the aforementioned disorders or for manufacture of a medicament for treating such disorders.

The efficacy of a nutraceutical or pharmaceutical composition of this invention in inhibiting the secretion of histamine or interleukin-4 can be evaluated by an in vitro assay well known in the art. See, e.g., Cheng et al., M. Taiwan J. Med., 1998, 3:166-173; and Cheng et al., J. of E.N.T. Medicine, 1998, 33: 431-441.

A nutraceutical composition of this invention can be further evaluated by clinical studies. For example, a group of patients suffering from allergic rhinitis (which is related to excessive excretion of histamine or interleukin-4 ) can be treated with the nutraceutical composition. Before the treatment, they exhibit some symptoms typical of allergic rhinitis including stuffy nose, sneezing, runny nose, itchy nose, itchy eyes, watery eyes, swollen eyes, and sore eyes. The patients are then orally administered with the nutraceutical composition, e.g., by a dose of 800 mg/10 kg/day, for an extended period of time, e.g., 14 days. Relief of the allergy can be observed, as characterized by reduced severity, and sometimes frequency, of the symptoms. Other symptoms that can be observed in the studies include nose and eyes rub, nose blow, carrying Kleenex, feeling embarrassed, thirst, feeling no well generally, tiredness, headache, scratchy throat, reduced outdoor activities, difficulties in sleeping at night, difficulties in concentrating, waking up during sleep, and limited daily activities. The frequencies of the symptoms before and after the treatment can be compared by statistical methods well know in the art, e.g., the Paired T-test.

Different dosages and administration routes can be tested. Based on the results, an appropriate dosage range and administration route can be determined.

Two tested nutraceutical compositions of this invention, which contained extracts of the whole plant of Centipeda minimal (L.) A. Br. Et Aschers and the flower of Magnolia lilifora Desr. (as the fourth herbs) as the fourth herb, respectively, proved to be efficacious in treating allergic rhinitis.

Without further elaboration, it is believed that one skilled in the art, based on the description herein, can utilize the present invention to its fullest extent. All publications recited herein are hereby incorporated by reference in their entirety.

### OTHER EMBODIMENTS

From the above description, one skilled in the art can easily ascertain the essential characteristics of the present invention, and without departing from the spirit and scope thereof, can make various changes and modifications of the invention to adapt it to various usages and conditions. Accordingly, other embodiments are also within the claims.

What is claimed is:

1. A nutraceutical composition comprising extracts of at least three or four herbs, wherein the first herb, which is optional, is Hedysarum polybotrys Hand.-Mazz. or Astragalus membranceus Bge., the second herb is Atractylodes macrocephala kodiz, the third herb is Ledebouriella seseloides (hoffm.) Wolff, and the fourth herb is an anti-allergy herb.

2. The nutraceutical composition of claim 1, wherein the fourth herb is Centipeda minimal (L.) A. Br. Et Aschers.

3. The nutraceutical composition of claim 2, wherein the weight ratio of the first, second, third, and fourth herbs is (0-1.5):(0.5-1.5):(0.5-1.5):(0.5-1.5).

4. The nutraceutical composition of claim 2, wherein the nutraceutical composition contains extracts of only four herbs, i.e., the first, second, third, and fourth herbs; or extracts of only three herbs, i.e., the second, third, and fourth herbs

5. The nutraceutical composition of claim 4, wherein the weight ratio of the first, second, third, and fourth herbs is (0-1.5):(0.5-1.5):(0.5-1.5):(0.5-1.5).

6. The nutraceutical composition of claim 1, wherein the fourth herb is Magnolia lilifora Desr.

7. The nutraceutical composition of claim 6, wherein the weight ratio of the first, second, third, and fourth herbs is (0-1.5):(0.5-1.5):(0.5-1.5):(0.5-1.5).

8. The nutraceutical composition of claim 6, wherein the nutraceutical composition contains extracts of only four herbs, i.e., the first, second, third, and fourth herbs; or extracts of only three herbs, i.e., the second, third, and fourth herbs.

9. The nutraceutical composition of claim 8, wherein the weight ratio of the first, second, third, and fourth herbs is (0-1.5):(0.5-1.5):(0.5-1.5):(0.5-1.5).

10. The nutraceutical composition of claim 1, wherein the nutraceutical composition contains extracts of only four herbs, i.e., the first, second, third, and fourth herbs; or extracts of only three herbs, i.e., the second, third, and fourth herbs.

11. The nutraceutical composition of claim 10, wherein the weight ratio of the first, second, third, and fourth herbs is (0-1.5):(0.5-1.5):(0.5-1.5):(0.5-1.5).

12. A method of treating a disorder related to excessive secretion of histamine, comprising administering to a subject in need thereof extracts of at least three or four herbs, wherein the first herb, which is optional, is Hedysarum polybotrys Hand.-Mazz. or Astragalus membranceus Bge., the second herb is Atractylodes macrocephala kodiz., and the third herb is Ledebouriella seseloides (hoffm.) Wolff, and the fourth herb is an anti-allergy herb.

13. The method of claim 12, wherein the fourth herb is Centipeda minimal (L.) A. Br Et Aschers or Magnolia lilifora Desr.

14. The method of claim 13, wherein the disorder is allergic rhinitis, asthma, or eczyma.

15. The method of claim 14, wherein the disorder is allergic rhinitis.

16. The method of claim 13, wherein the weight ratio of the first, second, third, and fourth herbs is (0-1.5):(0.5-1.5):(0.5-1.5):(0.5-1.5).

17. The method of claim 16, wherein the disorder is allergic rhinitis, asthma, or eczyma.

18. The method of claim 17, wherein the disorder is allergic rhinitis.

19. The method of claim 13, wherein the method does not include administering an extract of an additional herb.

20. The method of claim 19, wherein the weight ratio of the first, second, third, and fourth herbs is (0-1.5):(0.5-1.5):(0.5-1.5):(0.5-1.5).

21. The method of claim 20, wherein the disorder is allergic rhinitis, asthma, or eczyma.

22. The method of claim 21, wherein the disorder is allergic rhinitis.

23. The method of claim 12, wherein the disorder is allergic rhinitis, asthma, or eczyma.

24. The method of claim 23, wherein the disorder is allergic rhinitis.

25. The method of claim 12, wherein the method includes administering extracts of only four herbs, i.e., the first, second, third, and fourth herbs; or extracts of only three herbs, i.e., the second, third, and fourth herbs.

26. The method of claim 25, wherein the weight ratio of the first, second, third, and fourth herbs is (0-1.5):(0.5-1.5):(0.5-1.5):(0.5-1.5).

27. A method of treating a disorder related to excessive secretion of interleukin-4, comprising administering to a subject in need thereof extracts of at least three or four herbs, wherein the first herb, which is optional, is Hedysarum polybotrys Hand.-Mazz. or Astragalus membranceus Bge., the second herb is Atractylodes macrocephala kodiz., and the third herb is Ledebouriella seseloides (hoffm.) Wolff, and the fourth herb is an anti-allergy herb.

28. The method of claim 27, wherein the fourth herb is Centipeda minimal (L.) A. Br. Et Aschers or Magnolia lilifora Desr.

29. The method of claim 28, wherein the disorder is allergic rhinitis, asthma, or eczyma.

30. The method of claim 29, wherein the disorder is allergic rhinitis.

31. The method of claim 28, wherein the weight ratio of the first, second, third, and fourth herbs is (0-1.5):(0.5-1.5):(0.5-1.5):(0.5-1.5).

32. The method of claim 31, wherein the disorder is allergic rhinitis, asthma, or eczyma.

33. The method of claim 32, wherein the disorder is allergic rhinitis.

34. The method of claim 28, wherein the method includes administering extracts of only four herbs, i.e., the first, second, third, and fourth herbs; or extracts of only three herbs, i.e., the second, third, and fourth herbs.

35. The method of claim 34, wherein the weight ratio of the first, second, third, and fourth herbs is (0-1.5):(0.5-1.5):(0.5-1.5):(0.5-1.5).

36. The method of claim 35, wherein the disorder is allergic rhinitis, asthma, or eczyma.

37. The method of claim 36, wherein the disorder is allergic rhinitis.

38. The method of claim 27, wherein the disorder is allergic rhinitis, asthma, or eczyma.

39. The method of claim 38, wherein the disorder is allergic rhinitis.

40. The method of claim 27, wherein the method includes administering extracts of only four herbs, i.e., the first, second, third, and fourth herbs; or extracts of only three herbs, i.e., the second, third, and fourth herbs.

41. The method of claim 40, wherein the weight ratio of the first, second, third, and fourth herbs is (0-1.5):(0.5-1.5):(0.5-1.5):(0.5-1.5).
